(19) United States
(12) Patent Application Publication
(10) Pub. No.: US 2002/0192653 A1
(43) Pub. Date: Dec. 19, 2002

(54) Impedance-based chemical and biological imaging sensor apparatus and methods

(76) Inventors: Joseph Robert Stetter, Naperville, IL (US); William Roy Penrose, Naperville, IL (US)

Correspondence Address: Solomon ZAROMB, 9S 706 William Dr., Hinsdale, IL, 60521, US

(21) Appl. No.: 09/880,449

(22) Filed: Jun. 13, 2001

(51) Int. Cl. G01N 33/543

(52) U.S. Cl. 435/6; 382/128; 382/129; 436/151; 436/518

(57) ABSTRACT

A unique imaging sensor consists of a two-dimensional array of impedance electrode elements separated from chemical or biological samples contained in or containing fluids by a fluid-impervious layer which prevents electrode fouling. It can serve for various chemical and biological sensing applications, ranging from diagnosing cancer to monitoring metabolic changes in real time or the progress of cryosurgery. The imaging can be applied to single cells in a manner that allows only specific cell lines to attach [using selective derivatization of the layer surface], then monitors their viability, type, and status with impedance, and further measures their size and shape by use the two-dimensionality of the electrodes array.

### BACKGROUND OF THE INVENTION

This invention deals with a chemical and biochemical imaging sensor based on variations in electrical impedance.

Electrical impedance measurement has been applied to sensing in gas and liquid phases. It has also made inroads in physiology, medicine, bio-sensors, electronic instrumentation, electrochemistry, and analytical chemistry. The goals of such sensing ranges from diagnosing cancer to monitoring metabolic changes in real time or the progress of cryosurgery. Applications include complex approaches such as Electrical Impedance Tomography for 3-D impedance imaging of tissues and limbs as well as nano-technology arrays for sensing in single cells. Bio-sensors developed for food-borne pathogens can also use impedance to sense antigen-antibody reactions, e.g., to detect Staphylococcus enterotoxin B [SEB]. Field effect bio-sensors for fragments of DNA represent one use of impedance at the molecular level to monitor specific binding events.

Capacitance changes on short distance scales have been proposed and explored. U.S. Pat. No. 5,567,301, issued Oct. 22, 1996, to Stetter et al. and publications by Stetter et al., in the Technical Digest of the 5^{th} International Meeting on Chemical Sensors: Rome, Italy, Jul. 11-14, 1994, pp. 83-86 (Volume 1); Dr. D'Amico, Editor, University of Rome “Tor Vergata”, via Ricerca Scientifica, 06133 Rome, Italy; by DeSilva et al., in Biosensors and Bioelectronics, 10, 675-682, 1995; and by Feng et al., in Sensors and Actuators B, 35, 431-434, 1996, have described an unusual sensor made by sputtering a very thin film of platinum on a silicon oxide surface. This film was resistive and discontinuous, so that the channels and holes between “islands” of platinum served as a random array of capacitive elements. Binding of bio-molecules was detected by impedance spectroscopy. These sensors were difficult to make reproducibly.

Current practices for bio-detection using electrodes rely on placing them in contact with the cell or cellular material to perform sensing. To cite some examples, yeast is known to cause oscillations in pH that can be measured with electrical cell surface impedance sensing; AC impedance can be used to detect enzyme activity; interdigitated electrodes can be used to follow cellular growth because of impedance changes due to viable and non-viable cells; and a disposable field impedance-based bio-sensor has been reported for food pathogens. Disadvantages of current bio-sensors include fouling of the electrodes with extraneous matter. Sometimes impedance spectroscopy can be attempted to overcome the inteferences but one may still be left with fouling problems. Single cell flow cytometery coupled with fluorescence, light scattering, or impedance can be used to build immunotoxicological assays for a variety of target cell populations. Ultra-microelectrodes [carbon] were used to monitor the secretion from single rat melanotrophs.

Molecular detection, such as a urea sensor using bound urease to create NH₃ with subsequent reaction with HCl and conductivity change detection, illustrates the widespread use of conductivity or impedance approaches in chemical sensing.

### OBJECTIVES

It is an object of this invention to provide an improved chemical and biochemical imaging sensor for applications ranging from diagnosing cancer to monitoring metabolic changes in real time or the progress of cryosurgery or detection of specific gaseous or liquid species.

It is another object of this invention to provide a series of novel chemical and biochemical sensors with advanced capabilities, including (a) no fouling of the electrodes, (b) selectivity based on chemical or biochemical responses, and (c) physical imaging of the responses.

Still another object of the invention is to develop improved impedance-based methods for the detection of biological substances of interest and for the electro-analytical chemistry of impedance sensing.

It is a further object of the invention to provide an impedance imaging system for biological applications, such as sensing of SEB in contaminated foods, that is not susceptible to electrode fouling.

It is yet another object of the invention to provide an effective and simple imaging system for the detection of single cells, bioparticles, DNA fragments, and molecular specific events, such a system fulfilling some of the functions of an optical microscope besides providing specific chemical or biochemical information.

Conventional “sandwich” type immunoassay and other antibody-based sensing methods use fluorescent or chemiluminescent dyes to detect a secondary antibody layer which require expensive and delicate instruments. It is therefore still a further object of this invention to provide an inexpensive capacitor-array-based system that is effective within ranges of interest, and widely applicable to bio-sensing problems and biomedical applications, which permits antibody binding to be observed as it occurs, potentially speeding up the analysis process.

It is yet a further object of the invention to provide a capacitor-array-based system, wherein only glass or derivatized glass touches a test sample, there is no contamination of the sample, and influence upon the analyte is minimal.

It is still another object of the invention to provide an impedance-based system that could perform biochemical or biological imaging in vivo on living tissue.

A further object of the invention to provide an inexpensive impedance-based system comprising a multitude of miniaturized arrays on a single chip for nano-resolution in impedance space to get the shape of events in both spatial and temporal dimensions.

It is also an object of the invention to provide means and methods for simultaneous sensing of more than type of particle or substance by appropriate specific derivatizations to isolate specific molecular quantities or to achieve selective binding of bio-particles such as cells, spores, and pollen grains.

A still further object of the invention is to provide methods of interpreting quantitative impedance data in various media as well as impedance changes in terms of molecular or cellular functional parameters, such as presence of toxins, cell lines, viability, or metabolic changes.

### SUMMARY OF THE INVENTION

This invention is based on the discovery that impedance imaging of a sample having some electrical conductivity, such as chemical and biochemical samples contained in or containing liquids, is feasible even when the sensing electrodes are separated from the sample by a liquid-impervious layer which prevents electrode fouling. This discovery allows the use of a unique sensor consisting of a two-dimensional array of capacitive electrode elements for various chemical and biological sensor applications, such as selective binding and imaging of bio-particles such as cells, spores and pollen grains. The imaging can be applied to single cells in a manner that allows only specific cell lines to attach [using selective derivatization of the glass surface], then monitors their viability, type, and status with impedance, and further measures their size and shape by use the two-dimensionality of the electrodes array.

### BRIEF DESCRIPTION OF THE DRAWINGS

The invention is best explained with reference to the drawings in which:

FIG. 1 is a schematic of two cells of the capacitor array. FIG. 2 shows the gray scale response of the array of FIG. 1 to KCl solutions of different conductivities.

FIG. 3 shows a comparison of the impedance image of two approximately 90-μm-size corn pollen particles (FIG. 3A) with the corresponding optical image (FIG. 3B). The salt in the background buffer appears to gather around the pollen grains as it dries and brings about the observed contrasts in conductivity.

FIG. 4 shows the impedance image of 45-50 μm pecan pollen (FIG. 4A) and the corresponding optical image (FIG. 4B). Glycerol added to the background buffer prevents complete drying, so the pollen appears as light nonconductive particles on a dark conductive background.

FIG. 5 shows the image of conductive carbon particles dried in a nonconducting medium of Triton X-100 without salt, the carbon appearing as the conductive (dark) areas sensed by the array.

### DETAILED DESCRIPTION OF THE INVENTION

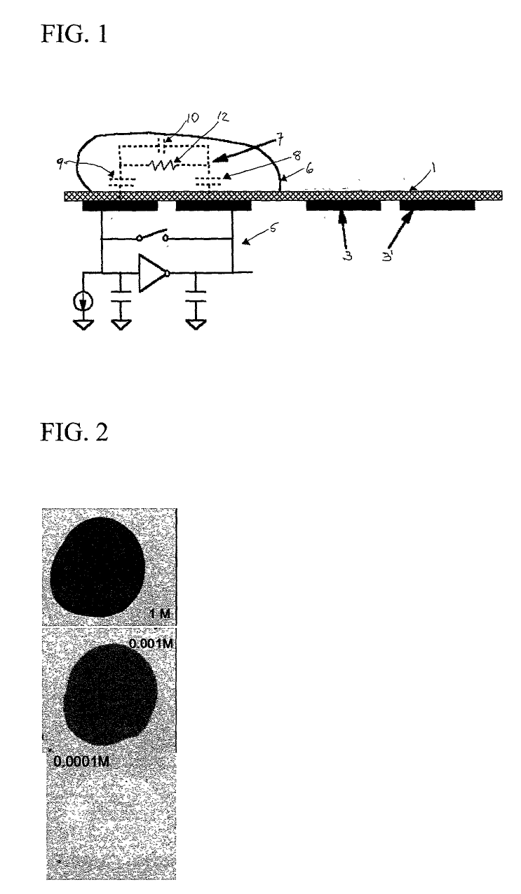

In the sensor shown in FIG. 1, an inert glass layer 1 separates an array of capacitive electrode pairs 3, 3′, each connected to an inverting amplifier circuit 5, from a conductive substance 6 whose equivalent circuit is indicated by the dotted-line schematic 7. The electrode pairs 3, 3′ are connected between the input and output of inverting amplifier 5 so as to yield a negative feedback. This sensor is similar to the capacitive distance sensor described in full detail in U.S. Pat. No. 6,114,862 of Tartagni et al., which was developed for fingerprint identification. The non-contact biosensor of FIG. 1 differs from that of the Tartagni patent by the presence of a conductive material 6 at the inert layer 1, which yields an equivalent circuit 7 comprising the virtual capacitors 8, 9, and 10, connected by a resistor 12, as indicated by dashed lines in FIG. 1.

The impedances of different parts of substance 6 are measured by appropriate electronic interrogation of each pair of electrodes 3, 3′, and each impedance value is assigned to an adjustable scale of 0 to 255 for display. With a single chip comprising an array of 256×364 electrode pairs, each 50×50 μm in size, the sensor can detect microscopic changes in impedance to about 10-20 μm from the outer surface of layer 1, reducing interferences from substances or particles in a bulk solution. Changes in the capacitance due to impedance changes at the outer surface of layer 1 are detected during interrogation of each capacitor pair 3, 3′ at a selected alternating current frequency, e.g., at 500 Hz.

Such an array has the advantage of a known geometry and reproducibility. Also, the electrical elements are isolated from the sample by the inert layer 1, comprising layers of glass, such as phosphosilicate glass, silicon nitride, and/or silicon carbide. Composite layers comprising a polymeric material, such as polyethylene, polypropylene, polymethacrylate polytetrafluoroethylene, or polycarbonate, may also be used. The isolation layer 1 also provides opportunities for the chemical attachment of active elements that can interact with biological molecules and particles.

FIG. 2 illustrates the grayscale change associated with KCl solutions of different concentration and hence different conductivity. The electric circuitry and software that services the array is similar to that disclosed in the afore-cited Tartagni patent and displays the output of the sensor array as a grayscale image with a resolution of 1:256. The grayscale image was calibrated by placing drops of KCl solution of different concentrations on the chip. Images of 1.0 M, 0.001 M, and 0.0001 M KCl solutions are shown in FIG. 2. The sensor measures the size, shape, and impedance of the drop. The grayscale values can be used to image conductivity at high resolution without direct contact, potentially in such difficult systems as the external surfaces of live organisms or cultured cells. Properties of physiological interest, such as concentrations of ions or molecular species, can be measured with this approach. For instance, urea can be sensed and measured by using bound urease to create NH₃ with subsequent reaction with HCl and conductivity change detection.

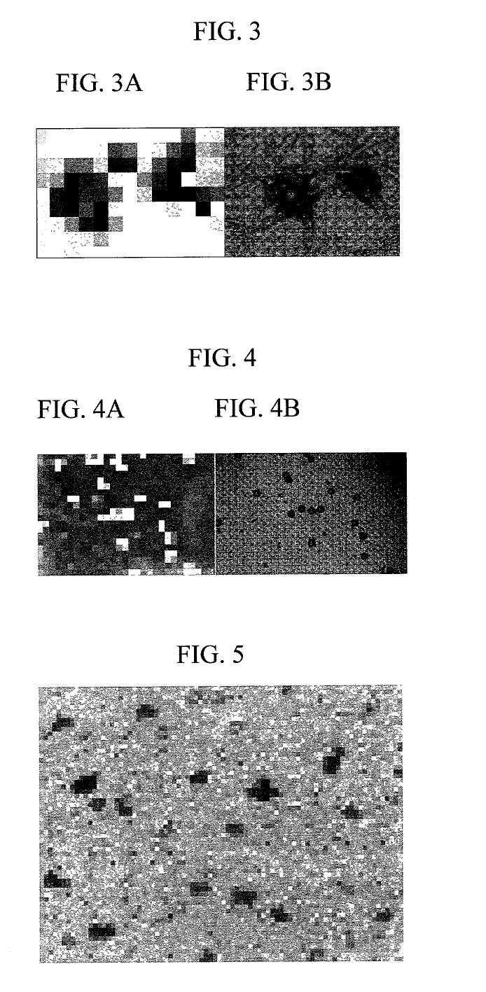

The sensitiveness of the sensor-array chip of FIG. 1 was tested with corn pollen dispersed over layer 1. We mixed the pollen in a salt solution and thus obtained the results of FIG. 3. By comparing the computer images from the sensor array with pictures of microscope images one can see the very sensitive response of the chip, which is expressed by the gray scale. FIG. 3 illustrates the grayscale identification of insulating bio-particles [pollens] in a conductive electrolyte. A comparison of optical and impedance images shows that both methods detect single particles or groups of particles.

In order to assess some of the potential chemical and biochemical applications, imaging experiments were performed with 90-μm corn pollen and 50-μm pecan pollen particles. The imaging chip does not appear to respond to dry pollen, but if the particles are suspended in dilute phosphate buffer and a trace of surfactant, the particles can be imaged in contrast. FIG. 3 shows optical and impedance images of corn pollen particles. The image of the corn pollen particles appeared from the suspension as it was drying. Salt particles formed about each pollen particle, creating a dark region of low impedance against the lighter, high-impedance background. The large corn pollen particles overlap more than one electrode pair element, or pixel, so that statistical analysis of the two-dimensional data could be used to estimate the average particle size. This is confirmed by the impedance images of the grains in FIG. 3.

FIG. 4 illustrates an alternative visualization technique using the smaller (˜50 μm) pecan pollen. Addition of 1% glycerol to the solution prevented complete drying and crystallization of the phosphate buffer, so that the background remained conductive (dark). The glycerol coated the particles making them non-conductive against the more conductive background. Therefore, where the pollen particles displaced the evenly drying solvent, their images appear as lighter shades of gray, which is a negative of the technique used to produce FIG. 3. Comparisons with optical images can be used for calibration and make the interpretation of the impedance images straightforward.

In FIG. 5, conductive carbon black particles (Vulcan XC-72) are imaged after deposition from a low-conductivity nonionic detergent solution in which they were suspended. Of course, these particles have very high conductivity and are imaged as the darker spots on the grayscale image.

A simple chemical sensor was prepared by mixing conductive carbon black and ordinary silicone vacuum grease. A thin layer of this mixture was coated on the outer surface 1 of FIG. 1. The image of the smear of the mixture was initially light colored, in fact, lighter than the background grey value of the sensor, which implies that the thin coated layer was non-conductive. Upon exposure to chloroform vapor, the image became considerably darker over a short time period, probably due to an increase in the number of conductive paths formed by the carbon particles caused by changes induced in the silicone-carbon layer upon solvent vapor exposure. This simple experiment demonstrates the potential of the capacitor array for chemical sensing applications.

Other materials are also expected to exhibit impedance pattern changes upon exposure to certain vapors, e.g, Nafion with water vapor.

The outer surface of the inert layer 1 can be chemically derivatized, and selectivity can be induced by the covalent binding of antibodies or oligonucleotides, using a procedure similar to that described in U.S. Pat. No. 5,567,301 of Stetter et al. and in the afore-cited publications by Stetter et al. (1994) and Feng et al. (1996). Mammalian cells, bacterial spores, and some live microorganisms are large enough to be visualized by the array in its present form. Quantitative measurements of ions in immediate proximity to cell membranes have been shown to be possible. Coating with reactive compounds may enable selective gas sensing. Because of the two-dimensional structure of the sensor, multiple channels of information can be acquired at one time.

Surface attachment of particles, such as pollen can be effectuated by activating the surface of layer 1 chemically and reacting with particles bearing amino groups on their surfaces. The chemically-attached particles should not be washed off or displaced by salt solutions, and should resist mild abrasion. This provides the basis for a generic type of biosensor that can be made selective to any type of bacteria, blood cell, or other biological particle by simply attaching an antibody that reacts with the particle of interest. To bring the binding phenomenon within the visible size range of the sensor array of FIG. 1, one immunochemical detection method makes use of a conventional “sandwich” type of assay. It consists of chemically attaching an antibody to the surface 1; capturing bacteria on the prepared surface; and reacting the surface with gold particles coated with more of the same antibody. The cells binding to the antibody on surface 1 may be smaller than the electrodes 3, 3′ and therefore not large enough to be seen with the array. However, their sides farthest from surface 1 will have their antibody-reactive groups still available to react with more antibodies which are attached to larger gold particles. If these are large enough, they become visible to the array, since gold is electrically conductive. Alternatively, an array of tinier pixels, e.g., 10 μm×10 μm or 1 μm×1 μm in size, or a disposition of a derivatized surface pattern directly in the gaps between electrodes 3 and 3′ of each capacitive pair, may yield improved imaging and resolution of smaller particles.

Thus, by selectively derivatizing the outer surface of layer 1 to allow only selected biological particles, such as cells, spores, pollen grains or other specific cell lines, to attach thereto, it becomes possible to monitor the size, shape, viability, type, or status of selected biological particles.

Simultaneous imaging, recognition, and quantitation of two or more different analytes, i.e., substances of interest, may be achieved by forming two or more differently derivatized surface layer patterns, each corresponding to a different analyte. Such multiple derivatizations may be effectuated by known masking and dipping or spin-coating techniques, preferably utilizing photolithography to achieve maximum resolution and precision. Each different pattern can be separately imaged by a programmed interrogation sequence.

It is thus clear that impedance changes are useful in the detection of chemical and biological substances. The molecular basis is easy to understand even if only pH changes or water activity changes are taken into account. The configuration of FIG. 1, which allows the sensing electrodes to be beneath the glass and yet the impedance near the glass surface to be controlling and thereby measured, can thus be seen to open the way to a broad range of chemical and biological sensing and imaging applications. Various gases or vapors which are hazardous or emanate from hazardous or illicit substances, such as chemical warfare agents, carcinogenic or otherwise toxic industrial emissions products, explosive compounds, or narcotics may thus be detected and possibly quantitated.

Outstanding examples of such applications are observations of molecular or cellular parameters, such as the toxins, cell lines, viability or metabolic changes, or detection of cancer cells or of food-borne pathogens. By comparing the images of observed cells with a computerized data base of cancerous cell shapes or of food pathogen shapes, it becomes possible to diagnose cancer or food contamination at computer speeds. Also, by following changes in impedance patterns, it becomes possible to perform biochemical or biological imaging in vivo on living tissue or on living cells or to monitor metabolic changes in real time or the progress of cryosurgery.

There will now be obvious many variations and modifications of the afore-disclosed embodiments to persons skilled in the art, e.g., changes in materials, in the shapes of the electrodes, in the insulating layer or the methods of electronic interrogation of the electrodes, which may yield improvements in sensitivity, selectivity, stability or response time . All of these variations and modifications will remain within the scope of this invention if defined by the following claims.

We claim:

1. A sensor for imaging chemical and/or biological samples contained in or containing fluids, which comprises:
an array of impedance electrode elements;
a fluid-impervious layer separating said elements from said samples so as to prevent electrode fouling and having an inner surface facing said elements and an outer surface on which said samples are placed; and
means for applying electrical interrogating signals to said elements, measuring impedance signals which are generated by said interrogating signals, and converting the resulting signals into visual images.

2. The sensor of claim 1, wherein said array is comprised of many tiny capacitive electrode pairs, each pair disposed as a tiny two-dimensional pixel, so as to form a two-dimensional imaging lattice.

3. The sensor of claim 2, wherein said fluid-impervious layer comprises phosphosilicate glass silicon nitride, silicon carbide, or a polymeric material, such as polyethylene, polypropylene, polytetrafl-uoroethylene, polymethacrylate or polycarbonate.

4. The sensor of claim 2, wherein a test sample can come in contact only with glass or derivatized glass, which causes no contamination of the sample, and whose effect on the sample is minimal.

5. The sensor of claim 1, wherein said outer surface is amenable to the chemical attachment of active elements that can interact with chemical or biological molecules and/or particles.

6. The sensor of claim 5, wherein said surface is treated so as to effectuate selective binding to said surface of specific molecular species or of specific biological particles.

7. The sensor of claim 6, wherein said treated surface permits antibody binding to be observed as it occurs.

8. The sensor of claim 2, wherein said two-dimensional pixels are small enough to permit detection and imaging of single cells, bioparticles, DNA fragments, and molecular specific events.

9. The sensor of claim 1, wherein said signals converting means is capable of acquiring two or more channels of information at one time.

10. The sensor of claim 1, wherein said signals converting means is capable of following changes in said samples so as to perform biochemical or biological imaging in vivo on living tissue or on living cells.

11. The sensor of claim 1, wherein said electrical interrogating, signals measuring, and signals converting means utilizes a selected alternating frequency for the interrogating signals and is able to interpret impedance changes in terms of molecular or cellular parameters.

12. The sensor of claim 1, wherein said signals converting means is able to effectuate quantitative measurements of ions in immediate proximity to cell membranes.

13. The sensor of claim 10, wherein said signals converting means comprises means for monitoring metabolic changes in real time or the progress of cryosurgery.

14. The sensor of claim 1, wherein said signals converting means comprises means for diagnosing any occurrence of cancer or of bacterial contamination by comparing observed images with a library of cancerous cell shapes or of pathogen cell shapes.

15. A method of imaging chemical and/or biochemical samples which comprises the steps of:
placing said samples adjacent to a fluid-impervious layer which separates them from an array of capacitive electrode elements;
applying electrical interrogating signals to said elements;
measuring impedance signals which are generated by said interrogating signals; and
converting the resulting signals in to visual images.

16. The method of claim 15, comprising a preliminary step of pre-treating the surface of said layer so as to effectuate selective binding to said surface of specific molecular species or of specific biological particles.

17. The method of claim 16, comprising the further step of interpreting said impedance signals so as to observe molecular or cellular parameters, such as the toxins, cell lines, bio-fouling or bio-materials buildup, viability or metabolic changes.

18. The method of claim 16, wherein said pre-treating step comprises derivatizing said surface to isolate specific molecular species.

19. The method of claim 18, wherein said pre-treating step comprises coating said surface with a reactive compound to enable selective gas or vapor sensing.

20. The method of claim 16, wherein said pre-treating step comprises selective derivatization of said surface allowing only selected biological particles, such as cells, spores, pollen grains or other specific cell lines, to attach thereto, and wherein said interpreting step comprises monitoring the size, shape, viability, type, or status of the attached biological particles.

21. The method of claim 20, wherein said pre-treating step comprises chemical derivatization of said surface and induction of selectivity by covalent binding of antibodies or oligonucleotides.

22. The method of claim 17, wherein said interpreting step comprises monitoring metabolic changes in real time or the progress of cryosurgery.

23. The method of claim 17, wherein said interpreting step comprises comparing observed images with a library of cancer cell shapes so as to diagnose any occurrence of cancer.

24. The method of claim 17, wherein said interpreting step comprises comparing observed images with a library of pathogen cell shapes so as to diagnose any occurrence of bacterial contamination.

25. The method of claim 21 which comprises generating two or more derivatized surface patterns, each pattern corresponding to a different analyte, and programming the sequence of interrogating signals so as to generate a separate image of each of said patterns.

26. The method of claim 19, wherein said gas or vapor is or originates from a hazardous or illicit substance, such as a chemical warfare agent, a carcinogenic or otherwise toxic industrial emissions product, an explosive compound, or a narcotic.

27. The sensor of claim 12, wherein said signals converting means is able to effectuate selective detection and measurements of specific molecular species.

28. The sensor of claim 27, wherein said species is absorbed or adsorbed from a gaseous phase.

29. The sensor of claim 28, wherein said species is or originates from a hazardous or illicit substance, such as a chemical warfare agent, a carcinogenic or otherwise toxic industrial emissions product, an explosive compound, or a narcotic.
